(19) United States
(12) Patent Application Publication
(10) Pub. No.: US 2020/0172908 A1
(43) Pub. Date: Jun. 4, 2020

(54) NANOCOMPOSITES FOR IMAGING AND DRUG DELIVERY

(71) Applicant: Microvascular Therapeutics LLC, Tucson, AZ (US)

(72) Inventors: Evan C. Unger, Tucson, AZ (US); Edmund Marinelli, Tucson, AZ (US); Delphine El Mehdi, Saint Louis, MO (US); Paul K. Olson, Saint Louis, MO (US)

(21) Appl. No.: 16/734,523

(22) Filed: Jan. 6, 2020

Related U.S. Application Data
(63) Continuation of application No. 14/430,186, filed on Mar. 20, 2015, now abandoned.
(60) Provisional application No. 61/703,607, filed on Sep. 20, 2012.

(51) Int. Cl. C12N 15/113; A61K 49/00; A61K 31/573; A61K 47/69; A61K 47/54; A61K 47/60; A61K 47/64; A61K 31/00; A61K 38/13

(52) U.S. Cl. CPC C12N 15/1136; C12N 2310/14; A61K 49/0054; A61K 49/0032; A61K 31/573; A61K 47/6911; A61K 47/6925; A61K 47/543; A61K 47/60; A61K 47/64; A61K 49/0082; A61K 31/00; A61K 38/13; C12N 2320/32; C12N 2310/3515; A61K 49/0093

(57) ABSTRACT

The invention provides a composition which includes a shell comprising at least one lipid, wherein said shell defines an enclosed space, a gas disposed within the enclosed space, and a coating of a polyalkylene glycol attached to and extending outwardly from lipid shell, wherein the lipid shell has a diameter from about 30 nanometers to about 5 microns.

### FIELD OF THE INVENTION

The invention is directed to nanoparticles comprising an outer stabilizing material bearing bioconjugates that target cell specific receptors. In certain embodiments, the nanoparticles are useful for diagnostic imaging and drug delivery

### BRIEF DESCRIPTION OF THE DRAWINGS

The invention will be better understood from a reading of the following detailed description taken in conjunction with the drawings in which like reference designators are used to designate like elements, and in which:

FIG. 1 illustrates one embodiment of Applicants' Nanocomposite (NC);

FIG. 2 illustrates Applicants' Nanocomposite (NC) comprising a targeting peptide, a fluorophore and a drug/prodrug;

FIG. 3A illustrates binding of NC and Internalization of NC by activated endothelial cells;

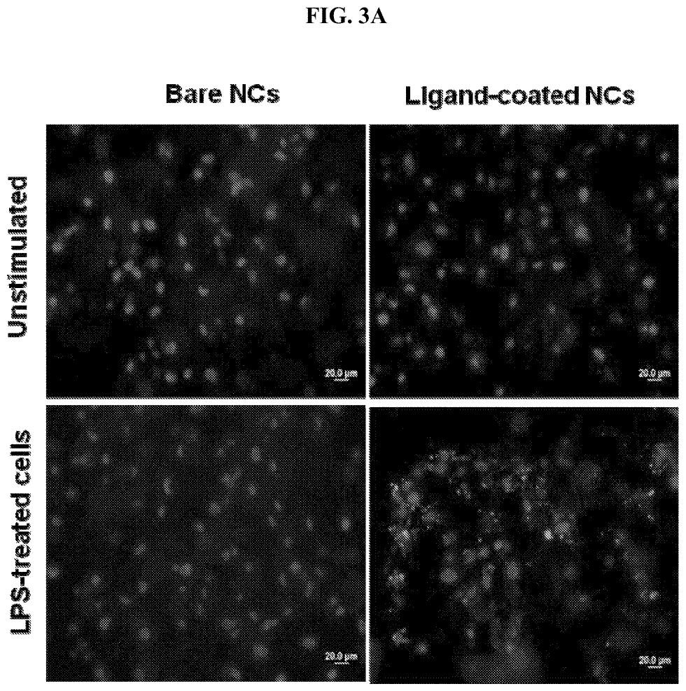

FIG. 3B is a higher resolution view of binding of NC and Internalization of NC by activated endothelial cells; and

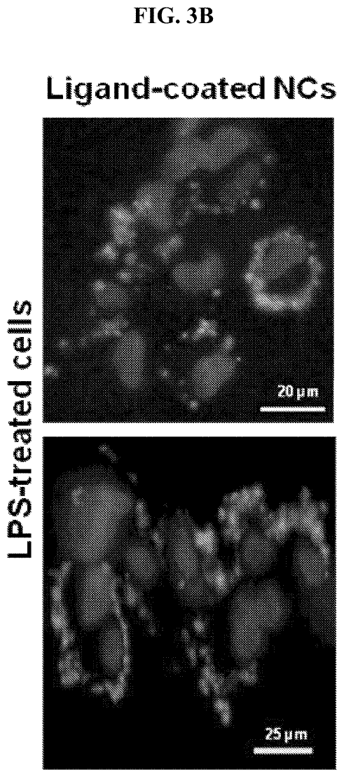

FIG. 4 shows ultrasound imaging of inflamed eye after injection of NC in rats.

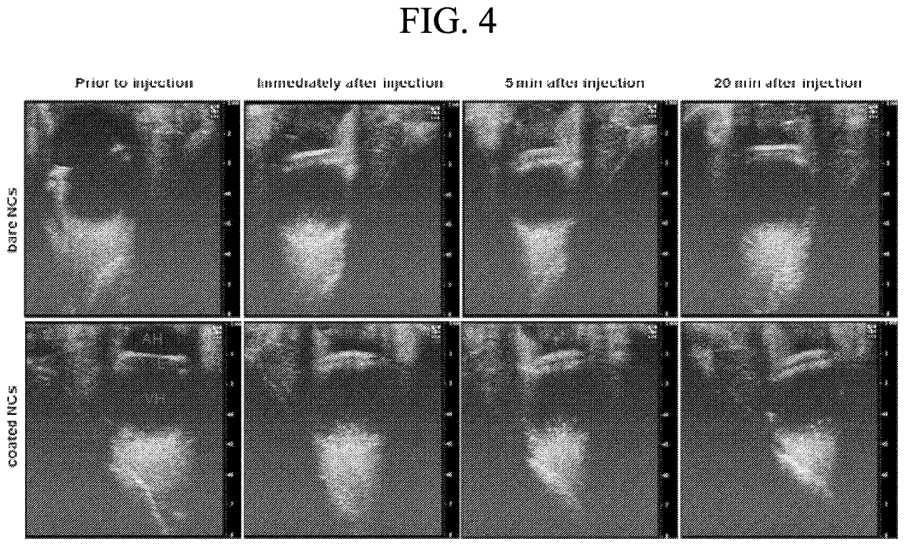

### DETAILED DESCRIPTION OF PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow charts included are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

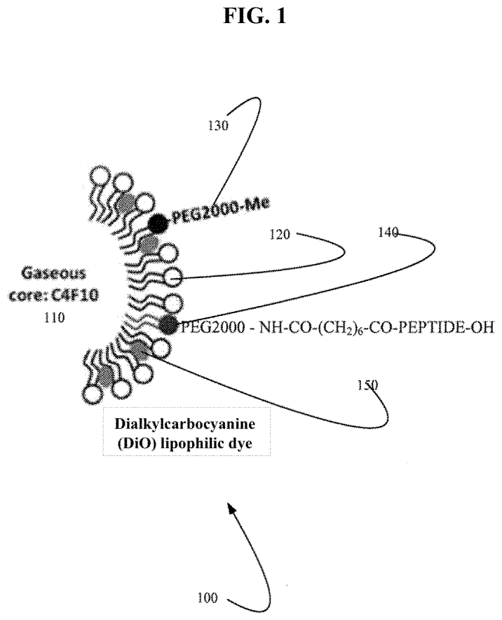

FIG. 1 illustrates the structure of one embodiment of Applicants' nanocomposite (“NC”) 100. In this case the NC 100 comprises a gaseous core 110. In certain embodiments, gaseous core 110 comprises perfluorobutane gas. NC 100 further comprises an outer stabilizing monolayer of phospholipid 120. A coating of polyethyleneglycol (PEG) 130 is attached to some of the phospholipid (2,000 Dalton molecular weight illustrated in FIG. 1). In certain embodiments, one or more peptides 140 may be affixed to the outer surface of the NC. In certain embodiments, a fluorescent dye 150, such as and without limitation a dialkylcarbocyanine, may be incorporated into the wall of the NC as well.

In certain embodiments, Applicants' NC may also comprise a drug or a prodrug adsorbed to the surface of the NC via electrostatic interaction with the charged moieties on the surface of the bubble. In other embodiments, Applicants' NC may also comprise a drug or a prodrug adsorbed to the surface of the NC by non-covalent interactions such as hydrophobic interactions with hydrophobic moieties on the surface of the NC.

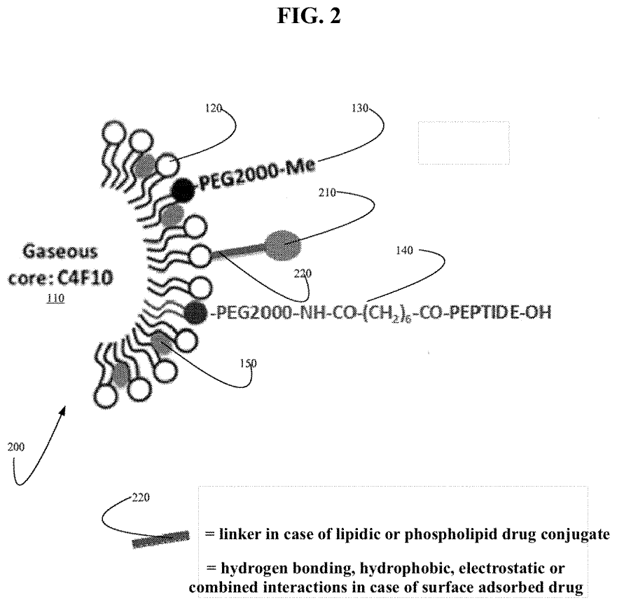

In still other embodiments, Applicants' NC may also comprise an active pharmaceutical Ingredient (“API”) adsorbed to the surface of the NC. By “API” Applicants mean a drug and/or a prodrug. In certain embodiments, the API is attached using hydrogen bonding interactions with moieties on the surface of the NC. In yet other embodiments, Applicants' NC may also comprise a drug or a prodrug adsorbed to the surface of the NC by the conjugation of a drug to a lipid or phospholipid whose long chain alkyl groups are then incorporated into the lipid membrane of the NC at the time of formation of the NC or after the formation of the NC. The general case of a drug or prodrug loaded NC is shown in FIG. 2.

Such a phospholipid-drug conjugate liberates the drug in vivo via cleavage of a linkage between the phospholipid and the drug in the physiological environment. Such a linkage can, for example, be chosen from ester, acyloxymethyl ester, amide, 2-thioalkylmaleimido, thioester, disulfide, amidine, imino, iminoether, N-Mannich bases (Drug-N—CH₂NH—C(═)—Ar, where Ar is an aromatic ring) for example.

The choice of the linker is dependant on the structure of the drug. For example, where the drug contains a hydroxyl group this hydroxyl group may be esterified with a phospholipid hemisuccinate ester to provide a mixed succinic ester of the drug and a phospholipid or lipid linked hydroxyl group. If the drug contains a carboxylic acid function the carboxyl group of the drug may be converted to a suitable amide derivative, phenolic ester, alkyl ester, thioester derivative or acyloxymethy ester derivative all of which are part of a lipidic or phospholipidic moiety which has the capacity to be incorporated into the membrane of the NC.

Upon binding of the NC bearing, optionally, a targeting peptide which homes to a site of disease via a specific biomolecular interaction, the NC can be imaged using fluorescence imaging (which can be performed either extra or intravitally) and/or ultrasound imaging instrumentation. If indeed there is a significant accumulation of NC at the site and this is judged to indicate the existence of disease at the site then the NCs may be subjected to higher power ultrasound that fluidizes, disrupts or even destroys the NCs with attendant local release of the drug in the physiological environment at the site of disease. The drug or prodrug may then be taken up by the cells via active or passive transport mechanisms.

Higher power insonation can serve to permeabilize the membranes of the cells in the diseased tissue as well. This can result in accelerated uptake of the drug or prodrug (described above) which was incorporated into the membranes or onto the surface of the NCs into the cells. In the case of the drug it exerts its effects either at the cellular membrane external to the cell if internalized as described above (via enhanced passive diffusion or by active transport) or at its intracellular target.

In the case of the prodrug, the prodrug may be processed by enzymes such as esterases or amidases in the physiological medium outside the cell or within the cell to give the drug whose fate is then the same as that of the drug described above.

Another path for utilization of the NC is the internalization of the entire NC by the targeted cells or by cells associated with the site of disease, injury and/or inflammation. In this case the NC, bearing the targeting peptide, optionally a fluorophore and optionally a drug adsorbed to the surface by electrostatic interactions, hydrophobic interactions, hydrogen bonding interactions, or as a conjugate of a lipid or phospholipidic-drug conjugate wherein the alkyl chain or chains of the lipidic or phospholipidic drug conjugate are situated in the membrane of the NC, are internalized by the cells at the site of disease or by other cells which have accumulated at the site of disease.

Cells that might be expected to accumulate at the site of disease, injury or inflammation are leukocytes, macrophages, T-cells, B-cells and dendritic cells. Macrophages in particular have a strong phagocytic function and as such those cells, besides the targeted cells of the diseased tissue, may specifically or non-specifically internalize NC. The NC may undergo intracellular trafficking and in the course of such trafficking the NC may shed their surface-adsorbed drug or prodrug which may in turn via intracellular trafficking be delivered to the desired intracellular target and exert the desired action. In the case of the prodrug it may be metabolically transformed into the desired drug in the intracellular environment, in the cytosol or in other intracellular compartments.

In the case of internalization of the NC by macrophages or other cells associated with the site of disease but which are not the diseased cells, higher power insonation (ultrasound irradiation) may allow sufficient disruption or permeabilization of the cellular structure such that the drug may escape such cells and enter the surrounding physiological fluid and then be taken up by the diseased cells to exert the desired action at the site of disease.

NC can also be used to detect inflamed and neovasculature in age-related macular degeneration (AMD). Diabetic retinopathy, uveitis, and other ocular disorders.

NC can also be used to detect inflammation in other disorders including: Ischemia reperfusion injury, trauma, diabetes, infection, cardiac arrest, myocardial infarction, stroke, sepsis, fever of unknown origin, acute respiratory distress syndrome (ARDS), multiple organ failure (MOF), COPD, traumatic brain injury (TBI), and asthma.

NC was prepared with dexamethasone palmitate and other steroid drugs such as Triamcinolone.

NC can also be prepared with palmitate or otherwise lipid/acyl-anchored versions of drugs. Other drugs would include, complement inhibitors (including members of the compstatin family), Immunosuppresive drugs such as cyclosporine, FK506, rapamicin, methotrexate, Anti vascular dugs such as VEGF inhibitors, PDGF inhibitors, FGF inhibitors, and Integrin inhibitors

The bioconjugate may vary from about 0.1 mole percent to about 10 mole percent of the wall forming lipids in the NC membrane. More preferably the bioconjugate ranges from about 0.5 mole percent to about 5 mole percent. Most preferably the bioconjugate is about 1 mole percent. More than one bioconjugate to a given target, e.g. E-selectin may be incorporated into the membrane.

Most preferably the targeting ligands is tethered to the surface of the NC with a hydrophilic polymer. The preferred hydrophilic polymer is polyethyleneglycol (PEG). The PEG chain may vary from 1,000 to 10,000 molecular weight, more preferably from about 1,000 to about 5,000 MW and most preferably is about 5,000 MW.

The targeting ligand preferably comprises a peptide but may also comprise a peptidomimetic material. The peptide may range from 4 to about 50 amino acids in length and may take the form of a monomer or a dimer. More preferably the peptide is from about 6 to about 20 amino acids in length.

The gas within the NC may comprise a fluorinated material, e.g. sulfur-hexafluoride, perfluoroethane, perfluoropropane, perfluorobutane, perfluoropentane, perfluorohexane, perfluoroheptane, perfluorooctane or mixtures thereof. More preferably the fluorocarbon material is perfluoropropane, perfluorobutane or perfluoropentane.

The lipids coating the NC may range in chain length from 12 to 22 carbon lengths with 16 or 18 carbon atoms preferred. The lipids may be saturated or unsaturated with the former preferred.

The NCs may range in diameter from 30 nanometers to 5 microns with NCs ranging from 100 nm to 2 microns in diameter more preferred.

The interior compartment of the NC may be filled with fluorocarbon material, water (e.g. aqueous material as in a liposome) or be filled with a crystalline material.

The bioconjugate is preferably directed to a receptor expressed on the surface of inflamed endothelial cells. The receptors include ICAM, VCAM-1, P-selectin and E-selectin. More than one ligand may target more than one receptor. The preferred target is E-selectin.

An example of a peptide that may be used in the NC is the E-selectin targeting dodecapeptide DITWDQLWDLMK-OH. Related peptides where L-methionine may be replaced with amino acids which contain isosteres for its side-chain sulfur can also be employed. Other atoms of the methionine side-chain may also be altered with isosteric moieties, which, besides mimicking the steric bulk of the methionine sulfur atom or another in the side chain, may provide an amino acid of greater stability to chemical transformation (such as oxidation) than possessed by methionine. Examples of such substitutions and the attribute(s) the substituted derivatives are expected to have are given in Table 1 below:

TABLE 1

| Methionine side | Isosteric amino | |
|---|---|---|
| chain atom | acid replacement | Expected |
| (bold) | side-chain atom | attribute(s) |
| CH₃— [symbol] —CH₂— | CH₃— [symbol] —CH₂— | Isosteric, stable to |
| | | oxidation |
| CH₃— [symbol] —CH₂— | CH₃— [symbol] —CH₂— | Isosteric, stable to |
| | | oxidation |
| CH₃— [symbol] —CH₂— | CH₃— [symbol] —CH₂— | Isosteric, stable to |
| | | oxidation |
| [symbol] — [symbol] —CH₂— | [symbol] — [symbol] —CH₂— | Isosteric, stable to |
| | | oxidation |
| CH₃— [symbol] —CH₂— | CH₃— [symbol] —CH₂— | Isosteric |
| CH₃— [symbol] —CH₂— | CH₃— [symbol] —CH₂— | Isosteric |
| [symbol] —S—CH₂— | [symbol] —S—CH₂— | Isosteric, S more |
| | | stable to oxidation |
| CH₃—S— [symbol] — | CH₃—S— [symbol] — | Isosteric, S more |
| | | stable to oxidation |
| [symbol] —S— [symbol] — | [symbol] —S— [symbol] — | Isosteric, S more |
| | | stable to oxidation |
| [symbol] — [symbol] — [symbol] [symbol] | [symbol] — [symbol] — [symbol] [symbol] | Isosteric, S more |
| | | stable to oxidation |
| [symbol] —S— [symbol] — | * [symbol] S—CH_{*}— | Isosteric, S more |
| | (thiirane ring) | stable to oxidation |

The peptide may comprise L or D amino acids or a mixture thereof. In addition to the modifications described above other amino acids of the dodecapeptide may be modified or substituted in a conservative or a non-conservative manner. As an example of a conservative substitution a tryptophan residue may be replaced with a 1-naphthylalanine residue, a 2-napthylalanine residue, a 2 or 3-benzothienylalanine, or a 2 or 3 benzofuranylalanine residue, or substituted tryptophan derivatives such as 5-hydroxytryptophan, 4,5,6,7-tetrafluorotryptophan and other benzene ring substituted tryptophan derivatives. Examples of non-conservative substitutions in the peptide sequence are replacement of a negatively charged side chain amino acid such as aspartic acid in the sequence with a positively charged side chain amino acid such as arginine, lysine or histidine. Other examples are replacement of an amino acid having a hydrophobic side chain with one having a hydrophilic and/or charged side chain. Replacement of a leucine in the sequence with an aspartic acid, lysine, threonine, glutamine, glutamate, asparagine or an arginine residue is illustrative of the concept. Such modifications, especially of residues that are not critical for binding, may be employed to increase the solubility, hydrophobicity or hydrophilicity of the peptide. The examples provided herein are illustrative and not limiting. Unnatural amino acids may also be employed to achieve the goal alterations of the binding or bulk physical properties of the peptide or NC beating the peptide. Peptidomimetic moieties may also be employed as replacements for one or more residues in the sequence. Such strategies are known to those skilled in the art. Such modifications may also be employed to alter the sign and magnitude of the zeta potential of the NC; such alterations of the zeta potential can reduce opsonization and recognition of the NC by the immune system, hence lengthening the blood half-time of the NC. Employing peptidomimetic moieties and/or D-amino acids at selected positions of the peptide results in stabilization of the peptide to the action of proteases or peptidases which can degrade the full sequence to shorter non-binding sequences. Improvements in the binding of the peptide to the target may also be obtained by substitution of L-amino acids in the targeting peptide sequence with D-amino acids. Also anticipated is the use of N-methyl amino acids at selected positions, where such substitution may increase the conformational flexibility of the peptide, increase the hydrophobicity of the peptide and stabilize the peptide to proteolytic degradation.

The subject peptides may be prepared by well established methods known to those skilled in the art. Typically the peptides are prepared by solid phase synthesis by either Boc chemistry or Fmoc chemistry those terms referring to the amino protecting groups employed on the alpha-amino group of side chain protected amino acids. The first residue bearing side chain and N-alpha protecting groups is appended to a resin which upon final deprotection provides the peptide as the C-terminal carboxylic acid and the N-terminus free for further manipulation as described in the examples below. After appendage of the first residue to the resin the N-alpha protecting group is removed; for Fmoc chemistry this requires treatment with 20-25% piperidine in DMF (dimethylformamide) for 5-20 min at ambient temperature followed by washing of the resin with DMF. In the case of Boc chemistry the resin is treated with trifluoroacetic acid to facilitate removal of the N-alpha Boc protecting group. The second amino acid bearing its N-alpha protecting group and side-chain protecting group is appended to the resin employing a peptide coupling agent such as diisopropylcarbodiimide with a coupling additive such as hydroxybenzotriazole (HOBt), or the combination of a more active coupling agent chosen from the phosphonium or uranium coupling agents such BOP or PyBOP (phosphonium coupling agents) or HBTU, HATU or TBTU, in the presence of a tertiary amine base such as diisopropylethylamine. Typically excess protected amino acid (4 equivalents), coupling agent (4 equivalents) and/or base (8 equivalents) with respect to the resin loading are employed. Coupling times can vary from as little as 2 min to as much as 3-6 or even 24 hours. In the case of Boc coupling protocols the N-alpha protecting group is the t-butoxycarbonyl group which is removed with trifluoroacetic acid (TFA) followed by washing of the resin with a tertiary amine base such as diisopropylethylamine. Coupling of the next and following amino acids is conducted as described for Fmoc chemistry. In the case of Fmoc chemistry the peptide is typically removed from the resin using treatment with TFA containing from 2-15% water and optionally containing additives that serve to scavenge reactive moieties generated by side chain protecting group cleavage. Examples of such scavengers are anisole, metacresol, thioanisole, triisopropylsilane and ethanedithiol. The peptide can be precipitated by pouring the deprotection mixture into cold methyl-t-butyl ether or cold diethyl ether. The precipitate is collected and subjected to analysis and purification by HPLC. In the case of Boc-chemistry the peptide is cleaved from the resin employing liquid hydrogen fluoride with a scavenger such as anisole (5-10%) at 0° C. for 45-60 min. The cleavage mixture is freed of HF by evaporation and the residue is triturated with ether to precipitate the peptide along with the resin. Then the residue is washed several times with ether and the residue is treated with 50% aqueous acetic acid to separate the peptide (which is now in solution) from the resin. As for peptides synthesized using Fmoc chemistry the crude peptide is purified by HPLC.

In the case where there interfering groups, such as amino groups not at the

N-terminus of the peptide, these are masked with orthogonal protecting groups. For example the C-terminal lysine amino group in the sequence DITWDQLWDLMK-OH can be protected with a protecting group which is stable to the conditions of cleavage of the other side chain protecting groups and cleavage of the peptide from the resin. The conjugation of the peptide to the phospholipid. PEG moiety is then followed by removal of the lysine N-epsilon protecting group to give the final product having the N-terminal lysine fully deprotected. Examples of such protecting groups are ivDdc [1-(4,4-Dimethyl-2,6-dioxocyclo-hexylidene)-3-methylbutyl] and Aloc (allyloxycarbonyl). The former protecting group is removed by treatment with 2% hydrazine in DMF, the latter protecting group can be removed using tetrakis triphenylphosphine palladium (0) in the presence of N-methylmorpholine in a mixed solvent such as chloroform/THF/acetic acid. The aloc group can also be removed employing resin bound palladium triphenyl phosphine and solid phase resin bound borohydride reagents under mild conditions. These methods described for synthesis and deprotection of peptides are known to those skilled in the art of peptide synthesis.

Treatment of human endothelial cells such as HUVEC (human umbilical vascular endothelial cells), HCMVEC (Human cardiac microvascular endothelial cells), and hREC (human retinal endothelial cells) with proinflammatory agents such as TNF-α, LPS (lipopolysaccharides) or IL-13 ‘activates’ the cells leading to an inflammatory cascade. Resultantly cell adhesion molecules are expressed in a temporal sequence. The first of these is P-selectin, then E-selectin followed by VCAM-1 and ICAM-1. P-selectin is expressed within minutes after stimulation of endothelial cells with LPS and its expression peaks 6 h whereas E-selectin expression peaks later followed by ICAM-1 and VCAM-1. P- and E-selectins mediate rolling of monocytes along the endothelial surface whereas ICAM-1 and VCAM-1 are involved with firm adhesion leading to extravasation of monocytes and leukocytes such as macrophages, processes which lead to release of cytokines and exacerbation of the inflammatory response.

A class of NC capable of both detection and quelling of the acute inflammatory response mediated early in the cascade, using a sufficiently long lived biomarker such as E-selectin, are expected to provide an efficacious and noninvasive method for management of acute inflammation such as that experienced in ocular conditions such as uveitis or endopthalmitis and other ocular disease conditions.

Surprisingly it was shown that NC with the composition shown above, not only bound to, but were internalized by human retinal endothelial cells (HREC) which had been ‘activated’ by treatment with proinflammatory agents such as lipopolysaccharide (LPS).

### EXAMPLE 1

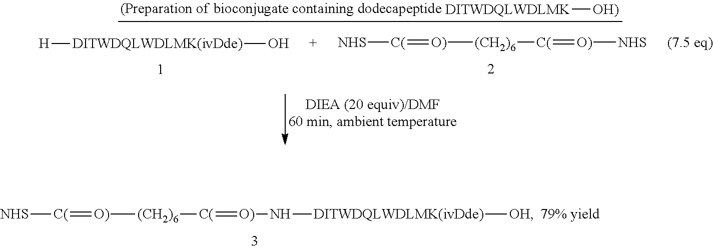

A 25 mL round-bottomed flask was charged with the peptide H₂N-DITWDQLWDLMK(ivDde)-OH (1) (0.073 g, 0.041 mmol), DMF (4.25 mL) and DIEA (0.310 g, 0.42 mL, 2.4 mmol, 58.5 equiv) and the mixture was stirred under nitrogen for 5 min. Most of the peptide was dissolved but a very small portion remained suspended. The mixture was put under high vacuum to remove volatiles. After about 10% of the solution volume was removed the solution clarified. After all of the volatiles were removed the resulting residue was dissolved in 1.3 mL of DMF and stirred. A separate 15 mL flask was charged with disuccinimidyl suberate (DSS) (2) (0.113 g, 0.3075 mmol, 7.5 equiv), DMF (1.3 mL) and DIEA (0.106 g, 0.14 mL, 0.82 mmol, 20 equiv) and the mixture was stirred. The solution of peptide 1 was aspirated into a pipette and added dropwise over 4 mm to the stirred solution of DSS/DMF/DIEA. Then a 0.9 mL portion of DMF was added to the flask originally containing the peptide solution and this was added dropwise over 2 min to the stirring mixture of DSS (2) and the peptide (1) in DMF/DIEA. The mixture was stirred 44 min and a 25 uL aliquot was withdrawn from the mixture added to a autosampler vial and the volatiles removed using a 30 mL BD syringe as an evacuation chamber. The residue was dissolved in 1.8 mL of acetonitrile-water 2/1 v/v (0.1% TFA) and subjected to HPLC analysis. This indicated consumption of the peptide 1 and formation of a new major product displaying a HPLC retention time (rt) of 25.12 min. The volatiles were removed from the mixture under high vacuum leaving a off-white residue in the flask. A 4 mL portion of acetonitrile was added and the entire content of the flask was transferred to a centrifuge tube and centrifugation was conducted for 5 min at 4000 rpm.

The supernatant was decanted and the process was repeated 3× using 3 mL aliquots of acetonitrile and centrifuging for 5 min at 4000 rpm. The resulting residue was pumped on at high vacuum for 10 min, then the pellet was broken up with a spatula and pumped on at high vacuum to complete dryness. This provided 65.6 mg (79.09% yield) of NHS-Sub-NH-DITWDQLWDLMK(iVDde)-OH (3), (Sub=suberoyl, NHS=N-hydroxysuccinimidyl) as a fine buff-colored powder. HPLC analysis of this material indicated the absence of DSS (rt 12.36 min) and the presence of the major product 3 in ca 89.4% yield (area %) (rt 25.14 min) accompanied by a small amount (2.25%) of a more hydrophobic product (rt 28.79 min) presumed to be the homodimer from reaction of 2 moles of the peptide 1 with DSS (2). The material was submitted for high resolution mass spectroscopic analysis. The ion series for the M+2H peak was consistent with the expected structure.

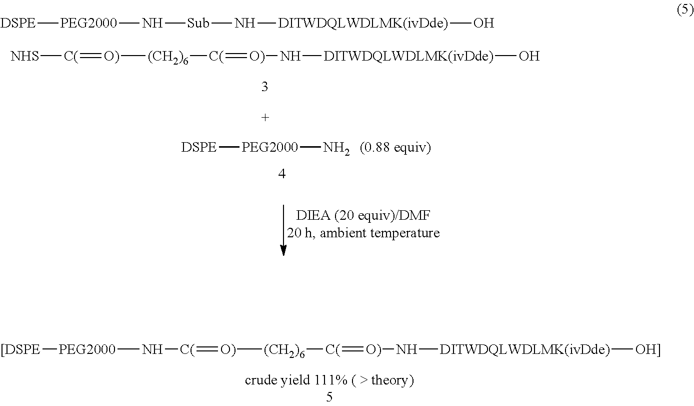

A 15 mL RB flask equipped with magnetic stir bar and septum cap was charged with DSPE-PEG2000-amine ammonium salt (4) (Avanti Polar Lipids) (0.086 g, 0.0308 mmol, 0.88 equiv) and N,N-dimethylformamide (5 mL) and N,N-diisopropylethylamine (0.181 g, 0.24 mL, 1.4 mmol, 40 equiv). The mixture was stirred 5 min and then the volatiles were evaporated at high vacuum over a period of 30 min to leave an off-white powder. Then DMF 1.0 mL and N,N-diisopropylethylamine (0.091 g, 0.12 mL, 20 equiv) was added to the powder and the mixture was stirred. A 71 mg (0.035 mmol, 1 equiv) portion of NHS-Sub-NH-DITWDQLWDLMK(iVDde)-OH (3) (89% pure) was dissolved in 1.0 mL of DMF and this added in one portion to the stirred solution of the DSPE-PEG2000-amine 4 and DIEA. A 1 mL volume of washings of the vessel containing the NHS-Sub-NH-DITWDQLWDLMK(iVDde)-OH (3) was added to the flask and the mixture was stirred at ambient temperature. After 20 hr the volatiles were removed at high vacuum to give 157 mg (>111% of theoretical yield) of a glassy residue. A ˜2 mg sample of the material was dissolved in ˜1.8 mL of 1/1 acetonitrile-water (10 mM triethylammonium acetate) and analyzed by HPLC using a Zorbax C3 column (4.6 mm id×250 mm, 300 angstrom pore, 5 micron particle). The eluent system was a linear gradient of 90/10 acetonitrile-water (10 mM triethylammonium acetate) into water (10 mM triethylammonium acetate) 40-90% over 45 min at 1 mL/min; detection UV at 290 nm. The chromatogram indicated the desired product 5 (89% area) at ret time 27.36 min. A minor product (˜11 area %) was noted at ret. time 39.2 min. This material is remaining DSPE-PEG2000-NH₂ (4) as indicated by its identical retention time in HPLC analysis.

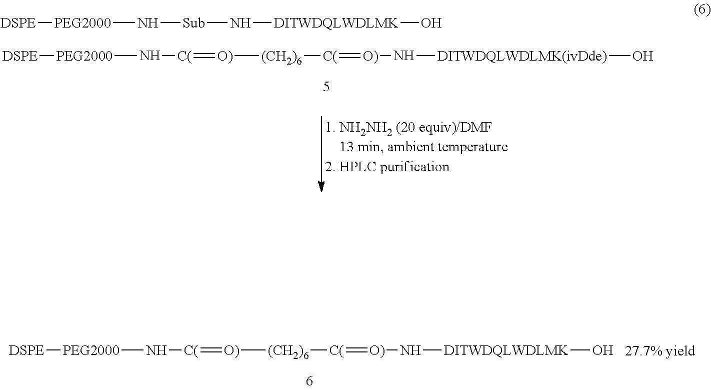

A solution of 4% hydrazine in DMF was made by adding neat hydrazine (200 uL, 204 mg, 6.375 mmol) to a 4.8 mL portion of DMF. This gave 5 mL of a 1.275 M solution of hydrazine in DMF. DSPE-PEG2000-NH-Sub-NH-DITWDQLWDLMK(ivDde)-OH (5) (0.0314 mmol, 147 mg) was added to a 15 mL round bottomed flask equipped with magnetic stir bar and septum cap and to this was added DMF (1.0 mL). The solution was mixed well and then a 1.04 mL portion of the hydrazine/DMF solution (42.2 equiv hydrazine) was added and the mixture was stirred for 13 min. Then the volatiles were removed at high vacuum (150 microns). A 25 uL aliquot of the reaction mixture was freed of volatiles and the residue was dissolved in 1/1 water/acetonitrile both containing 10 mM ammonium acetate (700 uL) and analyzed by HPLC. The major product appeared at a retention time of 27.74 min; this was the desired DSPE-PEG2000-NH-Sub-NH-DITWDQLWDLMK-OH (6). The mixture was kept on high vacuum for 3 hours and then under nitrogen for 2 days. The mixture was then dissolved in 25 mL of 40% acetonitrile-water (10 mM triethylammonium acetate). HPLC purification was conducted on a Zorbax 250 mm×9.4 mm i.d. C3 column (5 micron particle, 300 A pore). The crude mixture rich in compound 6 was applied to the column for 2 min at 4 mL/min and then the purification protocol was initiated. Eluants were: A—10 mM ammonium acetate, Eluent B—90% acetonitrile-water 10 mM ammonium acetate. The column was eluted at 4 mL/min with a linear gradient of 40-90% eluent B into eluent A over 45 min, then ramped to 95% B and held at 95% B to effect removal of any hydrophobic byproducts from the column and reequilibrated at 40% B. Pure product fractions were analyzed, combined, frozen and lyophilized to give 39 mg (27.7% yield) of the product 6 as a tacky lyophilizate. HPLC analysis indicated a purity of >98% and mass spectral analysis results were consistent with the expected structure.

### EXAMPLE 2

### Preparation of NCs shown in FIG. 1

DPPC (29.81 mg), DPPE-MPEG2000-sodium salt (5.61 mg), and DSPE-PEG2000-NH-Sub-NH-DITWDQLWDLMK-OH (6) (1.97 mg) were added to a 20 mL scintillation vial equipped with a magnetic stir bar and the vessel was charged with 3.9 mL of propylene glycol and set aside. A 100 mL beaker equipped with magnetic stir bar was charged with sodium chloride (244 mg), monosodium phosphate monohydrate (135.9 mg), anhydrous disodium phosphate (108 mg), glycerol (2.5 mL, 3.15 g) and nanopure water (42.5 mL). The mixture was stirred 6 min at ambient temperature to effect dissolution of all solids and mixing of all solvents. Both vessels were heated to 56-58° C. with stirring and a 1.1 mL aliquot of a solution prepared from 2 mg of DiO (3,3′ -dioctadecyloxacarbocyanine perchlorate) and 20 mL of propylene glycol was added to the scintillation vial containing the lipid mixture. The two vessels were stirred for 10 min followed by addition of the solution of dissolved lipids to the stirred aqueous buffered saline solution in four aliquots. Residual lipid solution was rinsed from the scintillation vial by addition of aliquots of the newly mixed buffer lipids solution to the scintillation vial, swirling and withdrawal of the solution from the scintillation vial and addition to the stirred buffer-lipids solution. After addition of the lipids solution to the buffered saline solution the mixture was stirred 10 min at 58° C., the beaker was covered with parafilm and the solution therein was allowed to cool to ambient temperature.

The resulting clear solution was aliquoted (1.5 mL) into 2 mL serum vials and each vial was fitted with a notched stopper depressed to half closure. The vials were then transferred to a crystallizing dish which was immediately transferred to a mini vacuum dessicator. The pressure was reduced to 75 mm Hg with a vacuum pump and then the dessicator was refilled with medical grade perfluorobutane. The dessicator was again evacuated to a pressure of 75 mm Hg and refilled as described. This procedure was repeated 4× after which the dessicator was opened and the stoppers fully depressed and crimp capped. The vials were stored at 4° C. until use.

### Preparation of E-selectin Targeting NCs Bearing the DiO Fluorophore

One of the 2 mL serum vials was removed from the refrigerator (4° C.) and allowed to warm to ambient temperature. The vial was then agitated for 45 seconds using a Lantheus Imaging Vial Mix agitator. Inspection of the vial indicated that about 40% of the volume of the vial consisted of NC and 60% of the volume was a highly turbid infranatant solution. The NC could be further segregated from the infranatant solution by centrifugation of the vial on a Sorvall Centrifuge using an HB-6 rotor at 1750 rpm (500 g) for 5 min. This gave a compacted layer of NCs and slightly turbid pale green infranatant. Aspiration of the vented vial to remove the infranatant and replacement of the infranatant with an equal volume of PBS followed by inversion of the vial and centrifugation as described above serves to remove excess lipids and DiO. Dilution of the NC (20-100 fold) and inspection by fluorescence microscopy disclosed the presence of fluorescent NCs with a narrow size distribution centered at ca 2 microns.

### EXAMPLE 3

### Incubation of NCs with Human Retinal Endothelial Cells Binding of NCs to Endothelial Cells and Internalization of NCs by Activated Endothelial Cells

Human retinal endothelial cells (HRECs) were pretreated with media alone or lipopolysaccharide (LPS) at 0.5 μg/ml for 5 hr. Before analysis, a flow of media or containing bare NC or ligands-coated NC was applied for 10 min (flow rate: 1 ml/min). Fluorescent microscopy, bar: 20 μum, Green=NC, blue: cell nuclei. Ligand-coated NC specifically binds to inflamed HRECs. Higher resolution shows that many NC are internalized in HRECs.

### EXAMPLE 4

### In Vivo Monitoring of NC Binding to the Inflammation Site in Inflamed Eyes

Rats received an intravitreal injection of LPS (10 ng) to trigger inflammation. After four hours, E-selectin-coated NC or bare NC were injected through the tail vein (1 ml/min). NC binding was monitored using microultrasound with high-frequency imaging equipment (Visual Sonics' Vevo® 2100 Imaging System, frequency 48 Hz). Asterisk shows the presence of NC at different time points after injection. Anatomical structures as shown in the transverse view of the inflamed eye: AH: aqueous humor, L: lens, VH: vitreous humor, R: Retina, OD: optic disk, ON: optic nerve. E-selectin targeted NC showed specific enhancement of inflamed retinal endothelial cells in rat eyes whereas control NC did not.

NC can also be used to detect inflamed and neovasculature in age-related macular degeneration (AMD). Diabetic retinopathy, uveitis, and other ocular disorders.

NC can also be used to detect inflammation in other disorders including: Ischemia reperfusion injury, trauma, diabetes, infection, cardiac arrest, myocardial infarction, stroke, sepsis, fever of unknown origin, acute respiratory distress syndrome (ARDS), multiple organ failure (MOF), COPD, traumatic brain injury (TBI), and asthma.

NCs were prepared with dexamethasone palmitatc and other steroid drugs such as Triamcinolone. NC can also be prepared with palmitate or otherwise lipid/acyl-anchored versions of drugs. Other drugs would include complement inhibitors (including members of the compstatin family), Immunosuppresive drugs such as cyclosporine, FK506, rapamycin, methotrexate, anti vascular dugs such as VEGF inhibitors, PDGF inhibitors, FGF inhibitors and Integrin inhibitors.

### EXAMPLE 5a

### Preparation of NCs Containing Dexamethasone Palmitate

NC with dexamethasone palmitate incorporated therein was prepared as follows. DPPC (21.83 mg), DPPE-MPEG5000-sodium salt (12.7 mg), and Dexamethasone palmitate (3.0 mg) were added to a 20 mL scintillation vial equipped with a magnetic stir bar and the vessel was charged with 5 mL of propylene glycol and set aside. A 100 mL beaker equipped with magnetic stir bar was charged with sodium chloride (244 mg), monosodium phosphate monohydrate (135.9 mg), anhydrous disodium phosphate (108 mg), propylene glycol (1.5 mL), glycerol (2.5 mL, 3.15 g) and nanopure water (41 mL). This mixture was stirred 6 min at ambient temperature to effect dissolution of all solids and mixing of all solvents. Both vessels were heated at 58° C. with stirring. Dissolution of the phospholipids was rapid (3 min) but the dexamethasone palmitate appeared to remain undissolved. The bath temperature for the scintillation vial was raised to 61° C. and stirring continued until the dexamethasone palmitate dissolved (˜20 min). During that time the beaker with the aqueous solution was removed from the water bath and covered with parafilm until the dexamethasone palmitate dissolved in the mixture stirred in the scintillation vial; it was then heated to 57° C. within 3 min. [Optionally an aliquot of a solution of DiO dye in propylene glycol was added to the mixture of lipids in the scintillation vial after the dexamethasone palmitate dissolved in order to provide NCs bearing targeting peptide, dexamethasone palmitate and a fluorescent tracer.] Then the solution of phospholipids and dexamethasone palmitate was added in two aliquots via pasteur pipette rapidly to the stirred aqueous solution. Residual lipids solution was washed into the stirring aqueous solution in the beaker by withdrawal of 2 mL aliquots from the aqueous solution, addition to the scintillation vial, agitation of the solution in the scintillation vial followed by transfer back to the aqueous solution. This was repeated 2×. After this the aqueous solution was stirred 5 min at 58° C. and then transferred into a 50 mL serum vial. The headspace was purged with dry nitrogen and the vial sealed by stoppering and crimp capping. The solution was stored 24 h at ambient temperature in the dark and then transferred to a refrigerator at 4° C.

The NC was prepared by aliquoting a 1.5 mL portion of the solution described directly above into a 2 mL serum vial. The vial was fitted with a notched stopper which was half depressed to allow for gas venting and entry. The vial head space gas was replaced with perfluorobutane as described in Example 2. The stopper was rapidly depressed and the vial was crimp capped. The vial was agitated for 45 seconds using a Lantheus Imaging Vial Mix. NC was washed as described 3×. For analysis NC were destroyed by addition of 1 mL of dimethylacetamide and 0.35 mL of propylene glycol followed by gentle vortexing and sonication in an ultrasonic cleaning bath. This gave a solution devoid of NC. HPLC analysis confirmed incorporation of dexamethasone palmitate in the NC.

### EXAMPLE 5b

### Preparation of E-selectin Targeting NC Containing Dexamethasone Palmitate

E-selectin NC with dexamethasone palmitate incorporated therein were prepared as follows. DPPC (21.83 mg), DPPE-MPEG2000-sodium salt (4.8 mg), DSPE-PEG2000-NH-Sub-NH-DITWDQLWDLMK-OH (6) (1.97 mg) and Dexamethasone palmitate (3.0 mg) are added to a 20 mL scintillation vial equipped with a magnetic stir bar and the vessel is charged with 5 mL of propylene glycol and set aside. A 100 mL beaker equipped with magnetic stir bar is charged with sodium chloride (244 mg), monosodium phosphate monohydrate (135.9 mg), anhydrous disodium phosphate (108 mg), propylene glycol (1.5 mL), glycerol (2.5 mL, 3.15 g) and nanopure water (41 mL). This mixture is stirred 6 min at ambient temperature to effect dissolution of all solids and mixing of all solvents. Both vessels are heated at 58° C. with stirring. Dissolution of the phospholipids is rapid (3 min) but the dexamethasone palmitate is dissolved by raising the bath temperature for the scintillation vial to 61° C. and stirring continued until the dexamethasone palmitate dissolves (˜20 min). During that time the beaker with the aqueous solution is removed from the water bath and covered with parafilm until the dexamethasone palmitate is dissolved in the mixture stirred in the scintillation vial; it is then heated to 57° C. within 3 min. [Optionally an aliquot of a solution of DiO dye in propylene glycol is added to the mixture of lipids in the scintillation vial after the dexamethasone palmitate is dissolved in order to provide NCs bearing targeting peptide, dexamethasone palmitate and a fluorescent tracer.] Then the solution of phospholipids and dexamethasone palmitate is added in two aliquots via pasteur pipette rapidly to the stirred aqueous solution. Residual lipids solution is washed into the stirring aqueous solution in the beaker by withdrawal of 2 mL aliquots from the aqueous solution, addition to the scintillation vial, agitation of the solution in the scintillation vial followed by transfer back to the aqueous solution. This is repeated 2×. After this the aqueous solution is stirred 5 min at 58° C. and then transferred into a 50 mL serum vial. The headspace is purged with dry nitrogen and the vial sealed by stoppering and crimp capping. The solution is stored 24 h at ambient temperature in the dark and then transferred to a refrigerator at 4 deg C.

The NC are prepared by aliquoting a 1.5 mL portion of the solution described directly above into a 2 mL serum vial. The vial is fitted with a notched stopper which is half depressed to allow for gas venting and entry. The vial head space gas is replaced with perfluorobutane as described in Example 2. The stopper is rapidly depressed and the vial is crimp capped. The vial is agitated for 45 seconds using a Lantheus Imaging Vial Mix. The NC are washed as described 3×. For analysis the NC are destroyed by addition of 1 mL of dimethylacetamide and 0.35 mL of propylene glycol followed by gentle vortexing and sonication in an ultrasonic cleaning bath. This gives a solution devoid of NCs. HPLC analysis confirms incorporation of dexamethasone palmitate in the NC.

### EXAMPLE 6

### Preparation of NCs Containing Dexamethasone as Liposomes

A blend of lipids as in Example 2 is prepared except that dioleoylphosphatidylcholine was substituted for the dipalmitoylphosphatidyl choline. The lipids are mixed in co-miscible solvent containing 13.5 mol % dexamethasone palmitate. The material is dried and rehydrated with normal saline and subjected to 5 freeze-thaw cycles. The material is then extruded against polycarbonate filters to yield 100 nm diameter liposomes with approximately 13.5% mole percent dexamethasone palmitate in the membrane of the liposomes

### EXAMPLE 7

### Preparation of Cationic NCs

NC is prepared as in Example 2 except that 10 mol % of the cationic lipid dimethyldioctadecylammonium (bromide salt), 18:0 DDAB, is incorporated into the membrane forming lipids. The cationic lipid 1,2-dipalmitoyl-3-trimethylammonium-propane (chloride salt) is similarly employed as the cationic lipid at 10 mol %.

### EXAMPLE 8

### Incorporation of siRNA Against VEGFs into Cationic NCs

Excess amounts of SiRNAs targeted to mVEGF-R2, mVEGF-R1 and mVEGF-A (Inhibition of ocular angiogenesis by siRNA targeting vascular endothelial growth factor pathway genes: therapeutic strategy for herpetic stromal keratitis Kim B, Tang Q, Biswas P S, Xu J, Schiffelers R M, Xie F Y, Ansari A M, Scaria P V, Woodle M C, Lu P, Rouse B T Am. J. Pathol. 2004 December; 6: 2177-85) are added to a suspension of cationic NCs prepared as described in Example 7. The vessel is inverted several times and allowed to stand 1 h at ambient temperature. The vessel is centrifuged in a Sorvall centrifuge using an HB-6 rotor at 1750 RPM (500 g) for 5 min. The vessel is removed and the infranatant solution is removed using a blunt-ended needle affixed to a syringe. A solution of PBS is added to the compacted NC and the vessel is gently inverted several times to allow free movements of the NC in the solution. The centrifugation procedure was repeated. Then the infranatant solution is withdrawn and replaced with PBS. This solution is employed for injection into the tail veins of mice for homing of siRNA carrying NC to the site of VEGF expression. Injection of the NC into mice with ocular angiogenesis followed by fluorescence imaging indicates accumulation at the site of angiogenesis. Insonation with ultrasound at a mechanical index of 0.1 to 0.4 followed by a waiting period indicates a reduction of angiogenic vasculature.

### EXAMPLE 9

### Treatment of Patient with Wet Macular Degeneration with siRNA NC

An ophthalmologist suspects wet macular degeneration in a patient and injects NC of Example 2 intravenously. Both ultrasound and fluorescence imaging are performed. Both imaging modalities show accumulation of NCs onto inflamed retinal vasculature. The ophthalmologist gives a second IV injection of NC from Example 2. Intermittently the ophthalmologist increases the power on the ultrasound probe from Mechanical Index (MI)=0.1 to MI=0.4. The siRNA is delivered into the inflamed endothelial cells and VEG-f expression is decreased. The damage from wet macular degeneration is improved. Note that the peak MI is 0.23 or less for opthalmological ultrasound imaging but might be increased for therapeutic applications.

While the preferred embodiments of the present invention have been illustrated in detail, it should be apparent that modifications and adaptations to those embodiments may occur to one skilled in the art without departing from the scope of the present invention as set forth herein.

1-20. (canceled)

21. A method for managing acute inflammation, comprising administering to a subject in need thereof a composition of nanocomposites comprising:
a lipid shell defining an enclosed space;
a gas disposed within the enclosed space, wherein the gas consists of one of perfluoropropane, perfluorobutane or perfluoropentane;
a coating of a polyethyleneglycol having a number average molecular weight from about 1,000 Daltons to about 5,000 Daltons attached to and extending outwardly from the lipid shell;
an active pharmaceutical ingredient attached to the lipid shell via hydrogen bonding or a linkage that is labile in vivo, wherein the active pharmaceutical ingredient is selected from the group consisting of an immunosuppressant, a VEGF inhibitor, a PDGF inhibitor, an FGF inhibitor, or an integrin inhibitor; and
a targeting ligand, configured to accumulate in vivo at a disease site, wherein the targeting ligand consists of a peptide ranging from 6 to 20 amino acids in length attached to the polyethyleneglycol;
wherein
the lipid shell comprises dimethyldioctadecylammonium, 1,2-dipalmitoyl-3-trimethylammonium-propane, dipalmitoylphosphatidyl choline or dioleoylphosphatidylcholine and has a diameter from about 100 nanometers to about 2 microns, and
the linkage is selected from the group consisting of an ester, acyloxymethyl ester, amide, 2-thioalkylmaleimido, thioester, disulfide, amidine, imino, iminoether, and N-Mannich base.

21. The method of claim 20, wherein the gas consists of perfluoropropane.

22. The method of claim 20, wherein the gas consists of perfluorobutane.

23. The method of claim 20, wherein the gas consists of perfluoropentane.

24. The method of claim 22, wherein the active pharmaceutical ingredient is selected from the group consisting of cyclosporine, FK506, rapamycin, dexamethasone, dexamethasone palmitate and methotrexate.

25. The method of claim 24, wherein the active pharmaceutical ingredient is dexamethasone palmitate.

26. The method of claim 24, wherein the linkage is an ester.

27. The method of claim 20, wherein the linkage is an acyloxymethyl ester.

28. The method of claim 20, wherein the linkage is a thioester.

29. The method of claim 24, wherein the peptide comprises dodecapeptide DITWDQLWDLMK-OH.

30. A method for managing acute inflammation of retinal vasculature, comprising administering to a subject in need thereof a composition of nanocomposites comprising:
a lipid shell defining an enclosed space;
a gas disposed within the enclosed space, wherein the gas consists of one of perfluoropropane, perfluorobutane or perfluoropentane;
a coating of a polyethyleneglycol having a number average molecular weight from about 1,000 Daltons to about 5,000 Daltons attached to and extending outwardly from the lipid shell;
an siRNA attached to the lipid shell via hydrogen bonding or a linkage that is labile in vivo, wherein the siRNA targets mVEGF-R2, mVEGF-R1 or mVEGF-A; and
a targeting ligand, configured to accumulate in vivo at a disease site, wherein the targeting ligand consists of a peptide ranging from 6 to 20 amino acids in length attached to the polyethyleneglycol;
wherein
the lipid shell comprises dimethyldioctadecylammonium, 1,2-dipalmitoyl-3-trimethylammonium-propane, dipalmitoylphosphatidyl choline or dioleoylphosphatidylcholine and has a diameter from about 100 nanometers to about 2 microns, and
the linkage is selected from the group consisting of an ester, acyloxymethyl ester, amide, 2-thioalkylmaleimido, thioester, disulfide, amidine, imino, iminoether, and N-Mannich base.

31. The method of claim 30, wherein the gas consists of perfluoropropane.

32. The method of claim 30, wherein the gas consists of perfluorobutane.

33. The method of claim 30, wherein the gas consists of perfluoropentane.
